(19) United States
(12) Patent Application Publication
(10) Pub. No.: US 2017/0181504 A1
(43) Pub. Date: Jun. 29, 2017

(54) A SELF-ATTACHING FABRIC AND METHODS OF MANUFACTURING SAME

(71) Applicant: ENFOLD TEXTILES LTD., Tel-Aviv (IL)

(72) Inventor: Meitar KARNI, Tel Aviv (IL)

(21) Appl. No.: 15/300,282

(22) PCT Filed: Mar. 30, 2015
(86) PCT No.: PCT/IL2015/050338

§ 371 Date: Sep. 29, 2016

(30) Foreign Application Priority Data
Apr. 7, 2014 (IL) 231984

(51) Int. Cl. A44B 18/00; D03D 27/06; D04B 1/16; D04B 7/20; D04B 25/00; D04B 21/14; D03D 11/02

(52) U.S. Cl. CPC A44B 18/003; D04B 21/14; D03D 27/06; D03D 11/02; D04B 7/20; D04B 25/00; D04B 1/16; A44B 18/0026; D10B 2501/0632; D10B 2331/04; D10B 2201/02; D10B 2201/04; D10B 2201/24; D10B 2211/02; D10B 2211/04

(57) ABSTRACT

The invention relates to a fabric which can attach to itself by use of hand pressure, and methods for its manufacture. The invention provides a fabric having at least part of its surface covered with small monofilament loops. The self-attaching fabric having at least part of its surface covered with small monofilament hooks which removably engage each other when the surface is pressed into contact with another similar surface to retain together the two portions of the fabric until they are separated by a peeling action.

### BACKGROUND OF THE INVENTION

The present invention relates to a fabric which can attach to itself by use of hand pressure, and methods for its manufacture.

More particularly, the invention provides a fabric having at least part of its surface covered with small monofilament hooks and loops.

The best known prior art is the original Velcro patent, U.S. Pat. No. 3,009,235. Use of this product entails adding two different pads to the surfaces to be joined. Velcro is not suitable for high fashion uses because of the bulge resulting when the two pads are in contact. It is also not versatile in positioning as the surfaces can only be joined in the area covered by both pads. In U.S. Pat. No. 8,551,596 Chou discloses self-sticking fabric where the hook and loop appear on opposing sides of the fabric. The structure is a simple jersey knit and the yarns which may be used are much limited by the need to chemically dissolve part but not all of the material used as hooks.

US Patent application 20130139555 shows a sleeve apparently made by warp knitting. The sleeve can be closed using the sewed-on Velcro strips.

Due to faulty translation and a lack of diagrams, patent CN101929020, which may he similar to the present invention, cannot be compared thereto.

### PRIOR ART

Prior art self-adhering fabrics result in a joint area which is too thick for high fashion use and too coarse for medical and infant use. They require sewing on of the joint materials and are limited to specific areas of the fabric.

### OBJECTS OF THE INVENTION

It is therefore one of the objects of the present invention to obviate the disadvantages of prior art self-attachment fabrics and to provide a fabric which can be joined at any area and by avoiding thick attachment areas can be used in high fashion garments.

It is a further object of the present invention to provide a soft fabric which can be used in goods designed for use of babies.

It is a further object of the present invention to provide a soft fabric which can be used in goods designed as functional clothing for disabled populations and for maternity wear

### SUMMARY OF THE INVENTION

The present invention achieves the above objects by providing a self-attaching fabric having at least part of its surface covered with small monofilament hooks and loops which removably engage each other when said surface is pressed into contact with another similar surface to retain together said two portions of said fabric until they are separated by a peeling action.

In a preferred embodiment of the present invention there is provided a fabric wherein said monofilament hooks and loops are made of transparent polyester yarn.

In a further preferred embodiment of the present invention there is provided a fabric wherein said hooks and loops are attached integral to the manufacturing process to a base fabric chosen from the group containing wool, cotton, viscose, silk, linen and blends thereof and others.

In another preferred embodiment of the present invention there is provided a fabric wherein a first part of its surface is covered with small monofilament hooks which removably engage a second part of its surface to enter the base fabric when pressed into contact therewith.

The invention also includes a manufacturing method comprising
STEP A: providing an industrial flat knitting machine having a front needle bed and a rear needle bed and being provided with electronic controls;
STEP B: loading said machine with yarns as needed to form the base fabric
STEP C: loading said machine with said monofilament yarn to form loops;
STEP D knitting the fabric including forming of said loops on at least one surface of said fabric
   and
STEP E selectively cutting some of the loops to form hooks.

In a further preferred embodiment of the method in STEP D further knitted rows are added to produce a thicker fabric having loops on both sides thereof.

It will thus be realized that the novel fabric of the present invention eliminates the need for external fasteners such as buttons, press studs, zippers and Velcro strips. These fasteners tend to cause unacceptable wear of delicate fabrics. For infants, the absence of buttons eliminates the danger of swallowing, and the bruising of delicate skin is avoided due to the absence of zippers and of Velcro strips. Thus there is an opening for use of the fabric for baby products such as bibs blankets and toys.

Due to the self-attaching characteristic of the said fabric, it may advantageously be used for the purpose of making clothing for disabled persons, in the way of functional clothing, with which the disabled person can more easily dress himself, thus lowering his dependency on others, and improving his quality of life.

It is also ideal for maternity wear where the item can be closed in varying locations thus adapting itself to the wearer's growing and changing proportions, fit to wear throughout the entire pregnancy and thereafter.

The fabric does not require any further brushing or any pile-raising process, thereby saving manufacturing cost, reducing the fabric thickness and thus widening the fields of applications.

It is easy to manufacture the fabric of the present invention with a uniform surface area, the loops covering the whole surface, or to limit knitting the loops to only a chosen section of the fabric.

The knitting machine can readily be programmed to knit some rows with and other rows without the monofilament loops.

The fabric of the present invention is ideal for use in garments where the qualities of “look and feel” are of importance. The small transparent hooks are visible only under close examination.

Furthermore, the versatile fastening properties of the fabric create a “one size fits all” benefit as the garment adapts itself to the shape of the wearer. Easy to close garments are also of use to persons suffering from incontinence, as the garment is more adaptable and useful for concealing worn absorption pads.

Experience has shown that wearers of a rectangular prayer shawl (Tallit”) repeatedly need to reposition this garment which slips off the shoulders during prayers. While a mechanical clasp, such as is described in Israel Patent 195828 is intended to solve this particular problem, the fabric of the present invention is ideal for retention of the Tallit in its required position, particularly for those having difficulty in operating a clasp.

Persons who are physically handicapped by Parkinson's, arthritis or have the use of one hand only may have much. difficulty in closing buttons, press-studs and zippers. However the task of getting dressed without outside help will be eased by clothing making use of fabric according to the present invention.

A further application of the present invention is in medical bandages, such bandages being closable without use of a safety pin.

### BRIEF DESCRIPTION OF THE DRAWINGS

The invention will now he described further with reference to the accompanying drawings, which represent by example preferred embodiments of the invention. Structural details are shown only as far as necessary for a fundamental understanding thereof The described examples, together with the drawings, will make apparent to those skilled in the art how further forms of the invention may be realized. In the drawings:

FIG. 1 is a diagrammatic representation of a preferred embodiment of the fabric while being manufactured, according to the invention, and

FIG. 2 is a representation of a further thicker embodiment thereof.

### DETAILED DESCRIPTION

The invention provides a fabric having at least part of its surface covered with small monofilament loops. These loops removably engage each other when said surface is pressed into contact with another similar surface to retain the two portions of said fabric. The two portions can be easily separated by hand gripping one of the portions and applying a peeling action. Attachment and separation can be carried out as often as desired. The structure of the fabric can best be understood from review of the method used in its manufacture.

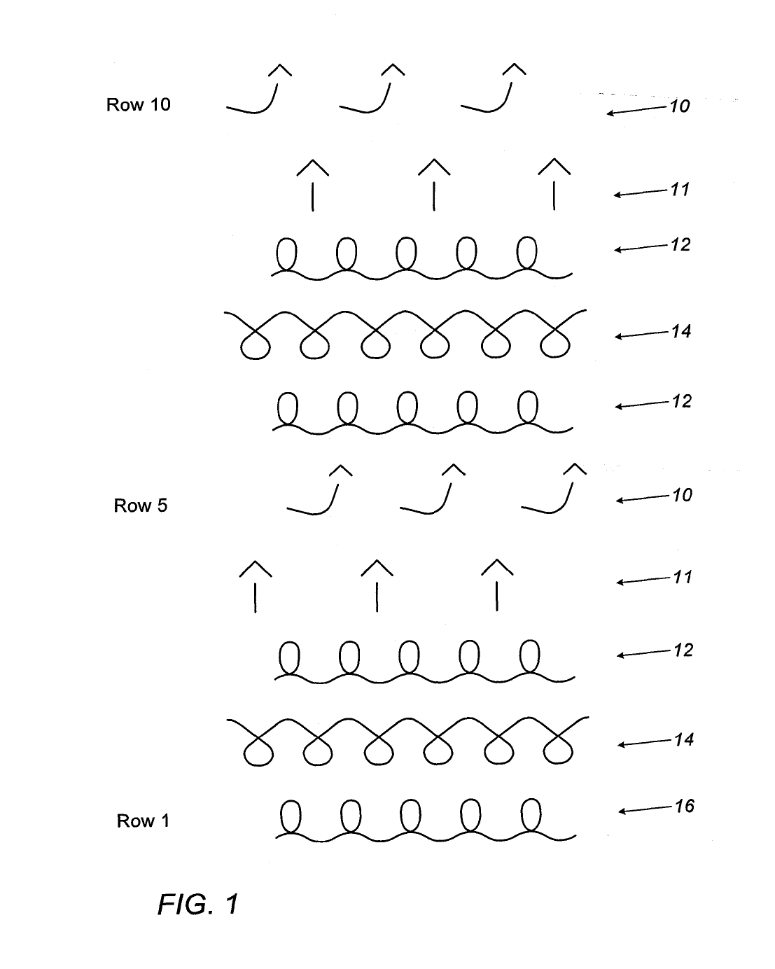

There is seen in FIG. 1 a representation of a method for manufacturing a first embodiment of the fabric, a thin fabric provided with hooks on one side only. The diagram refers only to STEP D, which in particular serves to reveal the structure of the resulting fabric.
STEP A: Providing an industrial flat knitting machine having a front needle bed and a rear needle bed and being provided with electronic controls. An example of a suitable machine is manufactured by STOLL, Germany. Typically the machines are for 12 gauge but multi-gauge machines are available from the same manufacturer.
STEP B: Loading the machine with yarns as needed to form the base fabric.
STEP C: Loading the machine with the monofilament yarn to form loops.

The description refers to the fabric sequence being performed on the rear needle bed, while the loops are knitted on the front bed. This arrangement can be reversed if desired.
STEP D knitting the fabric including forming of the loops on one surface of the fabric, as will be detailed herein.
STEP E cutting the loops to form hooks. There are various options for executing this step, for example inserting a long thin knife into the loops, as is explained in the Velcro patent, or cutting the loops externally using an array of heated knives mounted in a revolving roller, or using an oscillating laser beam. The present invention is not limited to any particular method for executing STEP E.

Referring now to STEP D and the diagram, the first and 6^{th} row represent dropping loops 10. The second and 7^{th} row represent transfer of alternate loops 10. The third, 5^{th} and 8^{th} row represent knitting using the yarns 12 comprising the base fabric. In the fourth and 9^{th} row the filament 14 is looped. Filament 14 is composed of polyester and second material of any kind or polyester as well. STEP D is completed in row 10 where a soft yarn 16 is knitted to retain the monofilament 14.

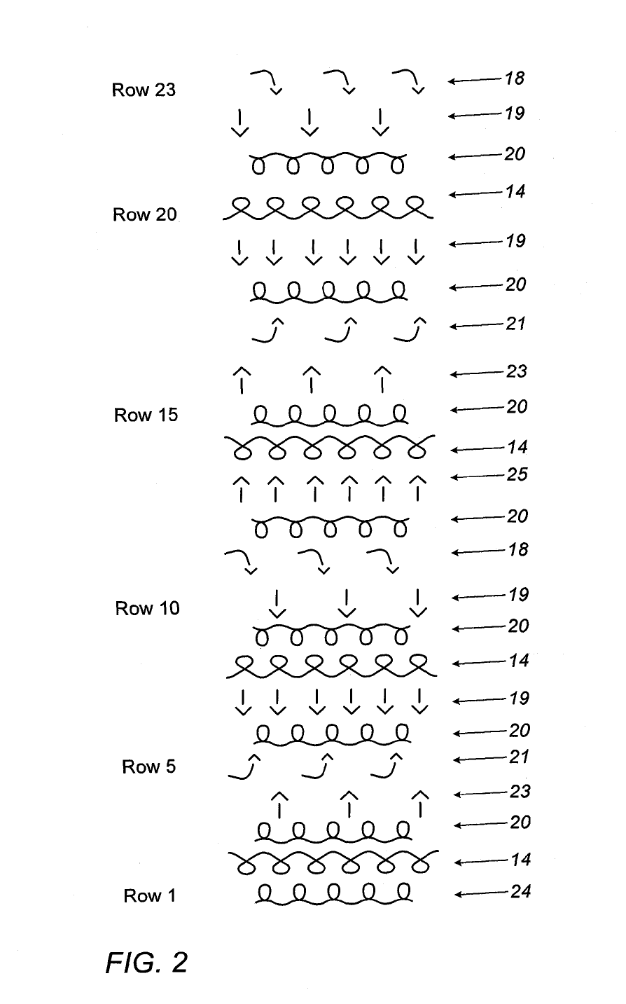

Referring now to FIG. 2, there is seen a representation of STEP D in a second embodiment wherein the fabric is provided with hooks and loops on both sides. This of course results in a fabric somewhat thicker than that described with reference to FIG. 1.

Row 1 of the diagram refers to release of alternate loops 18 from the rear needle bed (not shown). In row 2 the loops 18 are transferred 19 to the front bed (not shown).

Row 3 represents knitting of the basic yarn 22, while row 4 represents knitting the monofilament

In row 5 there is a transfer of loops 18 to the front bed, followed by knitting a row of the basic yarn 22 in row 6. Row 7 shows release 21 of loops 18 from the front bed, and their collection 23 by the rear bed is seen in row 8.

In row 9 there is a further row of knitting of the basic yarn 22, followed by knitting of the monofilament 14 in row 10.

In row 11 loops 18 of the monofilament are transferred 25 to the rear bed. In row 12 again the basic yarn 22 is knitted. Then in row 13 monofilament loops 18 are released from the rear bed and are transferred to the front bed in row 14. After knitting a further row of the basic yarn 22 in row 15, the filament 14 is again knitted in row 16. It is transferred to the front bed in row 17 and retained in row 18 by a row of basic yarn 22.

Some loops 18 are released from the front bed in row 19 for transfer to the rear bed in row 20. A further row of the basic yarn 22 is knitted in row 21. Finally a further row of the monofilament 14 is knitted in row 22 and is retained by a soft yarn 24 from the rear bed in row 23.

As the machine can be electronically programmed it is obvious that many variations of the described embodiments can be produced by adding or subtracting rows or by changing the order thereof, such as one side of the fabric having polyester loops while the other side small loops of wool or other material. Alternatively, both sides of the loops are of polyester.

Therefore the scope of the described invention is intended to include all embodiments coming within the meaning of the following claims. The foregoing examples illustrate useful forms of the invention, but are not to be considered as limiting its scope, as those skilled in the art will be aware that additional variants and modifications of the invention can readily be formulated without departing from the meaning of the following claims.

We claim:

1. A self-attaching fabric having at least part of its surface covered with small monofilament hooks and loops which removably engage each other when said surface is pressed into contact with another similar surface to retain together said two portions of said fabric until they are separated by a peeling action.

2. The fabric as claimed in claim 1, wherein said monofilament hooks and loops are made of transparent polyester yarn.

3. The fabric as claimed in claim 1, wherein said hooks and loops are attached integral to the manufacturing process to a base fabric chosen from the group containing wool, cotton, viscose, silk, linen and blends thereof or others.

4. The fabric as claimed in claim 1, wherein a first part of its surface is covered with small monofilament hooks which removably engage a second part of its surface to enter the base fabric when pressed into contact therewith.

5. The fabric as claimed in claim 4, wherein the filament is composed of two filaments.

6. A method for manufacturing the fabric as claimed in claim 1:
STEP A: providing an industrial flat knitting machine having a front needle bed and a rear needle bed and being provided with electronic controls;
STEP B: loading said machine with yarns as needed to form the base fabric
STEP C: loading said machine with said monofilament yarn to form loops;
STEP D knitting the fabric including forming of said loops on at least one surface of said fabric, and
STEP E selectively cutting some of said loops to form hooks.

7. A method as claimed in claim 5, wherein STEP D comprises
STEP D1 knitting a single row of the base fabric yarn on needles of said rear needle bed;
STEP D2 knitting tucks of monofilament yarn on all needles on said front needle bed while knitting regular stitches on said rear bed;
STEP D3 knitting a further single row on said rear bed;
STEP D4 transferring alternate stitches of monofilament yarn from said front bed to said rear bed
STEP D5 forming monofilament loops by dropping all remaining monofilament stitches remaining on said front bed, and
STEP D6 repeating STEPS D1-D5, while in STEP D4 the stitches to be transferred are those stitches which were not transferred previously in STEP D4.

8. A method as claimed in claim 6, wherein STEP D4 comprises transferring all stitches of monofilament yarn from said front bed to said rear bed.

9. A method for manufacturing the fabric as claimed in claim 5, wherein in STEP D further knitted rows are added to produce a thicker fabric having loops on both sides thereof.
